(19) United States
(12) Patent Application Publication
(10) Pub. No.: US 2005/0101579 A1
(43) Pub. Date: May 12, 2005

(54) Endometriosis treatment protocol

(76) Inventor: Eugene R. Shippen, Wyomissing, PA (US)

Correspondence Address: Richard C. Litman;LITMAN LAW OFFICES, LTD., P.O. Box 15035, Arlington, VA, 22215, US

(21) Appl. No.: 10/982,915

(22) Filed: Nov. 8, 2004

Related U.S. Application Data
(60) Provisional application No. 60/517,388, filed on Nov. 6, 2003.

(51) Int. Cl. A61K 31/59; A61K 31/57

(52) U.S. Cl. 514/167; 514/170

(57) ABSTRACT

The endometriosis treatment protocol provides for administering to a female patient in need of treatment for endometriosis a pharmaceutical composition in a form suitable for vaginal or rectal delivery having a pharmaceutically effective amount of an aromatase inhibitor, which may be either a steroid or non-steroidal. The pharmaceutical composition may be formed as a vaginal suppository, a rectal suppository, a vaginal gel, a rectal gel, a vaginal cream or a rectal cream. The pharmaceutical composition may optionally have pharmaceutically effective amounts of progesterone and calcitriol, and may be administered in combination with an oral COX-2 inhibitor. Alternatively, the pharmaceutical composition comprises an aromatase inhibitor administered vaginally or rectally and is administered in combination with oral calcitriol and the oral COX-2 inhibitor. The aromatase inhibitor is either steroidal or non-steroidal.

### CROSS-REFERENCE TO RELATED APPLICATION

This application claims the benefit of U.S. Provisional Patent Application Ser. No. 60/517,388, filed Nov. 6, 2003.

### BACKGROUND OF THE INVENTION

1. Field of the Invention

The invention relates to treating endometriosis using a vaginal or rectal suppository, cream or gel comprising a pharmaceutically effective amount of an aromatase inhibitor.

2. Description of the Related Art

Endometriosis is a progressive disease affecting up to about ten percent of women after the onset of menstruation. A diagnosis can be made by laparoscopy. Medical treatment designed to interfere with ovulation generally provides effective pain relief. However, current treatments for endometriosis are plagued with unacceptable side effects and high failure rates.

Endometriosis is probably initiated by reverse spillage of normal endometrium (eutropic endometrium) through the fallopian tubes into the abdominal cavity where endometrial cells adhere to the surfaces of the abdominal contents to form endometrial implants, i.e. ectopic endometrium. The presence of endometrial implants on abdominal surfaces initiates an inflammatory reaction that leads to symptoms such as pain and cramping, particularly around the time of menstruation. The symptoms tend to get worse under the repetitive influence of the menstrual cycle and may become continuous throughout the menstrual cycle.

Endometrial cells start producing an enzyme called aromatase, which allows the endometrial cells to begin self-generating estrogens, estradiol and estrone. This enzyme is not normally present in eutropic endometrium. It is unknown why the aromatase enzyme is activated in endometrial cells, but genetic factors may be involved. It is known, however, that inflammation may up-regulate aromatase activity and, therefore, may be the primary activator of aromatase expression within endometrial cells.

Estrogens can be made from other hormones circulating in the body, such as adrenal androgens, DHEA and androstenedione or from ovarian testosterone through the action of aromatase. This ability to internally produce estrogens allows the endometrial implants to sustain and increase growth. Additionally, estrogens are known to up-regulate the inflammatory process. This increase in the production of inflammatory substances then further increases the aromatase enzyme activity in a forward feedback loop.

The estrogen produced by the endometrial implants increases inflammation, a primary factor in the symptomatology of the disease. Specifically, inflammation is induced by estrogens locally within the implants through up-regulation of the COX-2 pathway, resulting in production of prostaglandins. These prostaglandins recruit immune cells, such as lymphocytes and macrophages, which infiltrate the areas of the implants, leading to further inflammatory responses. The prostaglandins also help establish the implants by inducing growth of blood vessels that carry nutrients to the implants, encouraging further growth of the implants and causing the disease to become progressively worse over time.

Treatments for endometriosis have been focused on reducing production of estrogen by the ovaries or blocking estrogen receptors within the endometriosis cells or implants. Such treatments include the use of high doses of progesterone or synthetic analogues thereof (progestins or progestagens), Gonadotropin releasing hormone (GnRH) agonists, and analogs thereof, and danazol. Both high dose progestins or progestagens and Gonadotropin releasing hormone (GnRH) agonists, and analogs thereof, block the release of the reproductive gonadotrophins LH (luteinizing hormone) and FSH (follicle stimulating hormone) causing the ovaries to shut down and stop producing estrogen, thereby reducing the estrogenic drive to sustain the endometrial implants for prolonged periods. Such treatments are able to negate at least some of the effects of ovarian estrogen on the implants and thereby cause the implants to regress. However, none of the treatments are effective in reducing the self-generation of local estrogen production within the implants from adrenal androgens, DHEA and androstenedione. These treatments are poorly tolerated by many women and frequently fail to clear the endometrial implants completely, and symptoms often recur.

Danazol is a synthetic testosterone derivative that is sometimes prescribed to lower estrogen production and shrink endometriosis lesions in the abdomen. Danazol has a high side effect profile, including acne and weight gain that has rendered the treatment less acceptable to women. It rarely results in sustained remission of symptoms or growth of the implants.

Surgical laser ablation is sometimes used when medical treatments fail. The success rate of the laser treatment varies from patient to patient according to the relative accessibility of the endometrial implants to the laser. If the laser surgery is unable to remove all the endometrial implants the symptoms often recur and remain problematic as long as menstruation persists.

International Patent Number WO 02/072106 (“the WIPO '106 patent”), published Sep. 19, 2002, discloses a combined synergistic method for preventing and treating hormone dependent disorders, such as endometriosis. The method comprises the step of administering to a patient in need of such treatment an effective amount of aromatase inactivator exemestane (itself disclosed in, e.g., U.S. Pat. No. 4,808,616), alone or in combination with additional therapeutic agents to provide a synergistic effect. Examples of additional therapeutic agents include: Danazol; a COX-2 inhibitor, such as rofecoxib; a non-steroidal anti-inflammatory compound (NSAID), such as acetyl salicylic acid; a retinoid compound; a matrix metallo-protease inhibitor; an anti-estrogen compound; a GnRH agonist or antagonist; a selective progestins receptor modulator (SPRM); and an angiogenesis inhibitor, or a mixture thereof. The WIPO '106 patent does not teach or suggest using a vaginal or rectal suppository, gel or cream to obtain exceptional results in the treatment of endometriosis as found in the present invention. The WIPO '106 patent also teaches away from the present invention, since exemestane was regarded as a medicine taken by mouth. Specifically, the WIPO '106 patent did not teach administering a composition comprising exemestane to a patient via, for example, a vaginal suppository.

U.S. Pat. No. 5,166,200, issued Nov. 24, 1992 to Fujise et al., describes a remedy for endometriosis comprising, as an active ingredient, 14α-hydroxy-4-androstene-3,6,17-trione or an ester derivative thereof. The '200 patent states that the remedy can effectively treat endometriosis by the strong aromatase-inhibitory activity without giving serious side effects. The remedy compounds are made into suitable pharmaceutical preparations for administering to a patient, such as an oral agent, suppository, injection and the like. The '200 patent does not teach or suggest using a synergistically effective pharmaceutical composition of the present invention in the form of a vaginal or rectal suppository, gel or cream to obtain exceptional results in the treatment of endometriosis.

None of the above described drugs and patents, taken either singly or in combination, are seen to describe the instant invention as claimed. Thus, an endometriosis treatment protocol solving the aforementioned problems is desired.

### SUMMARY OF THE INVENTION

The endometriosis treatment protocol provides for administering to a female patient in need of treatment for endometriosis a pharmaceutical composition in a form suitable for vaginal or rectal delivery comprising a pharmaceutically effective amount of aromatase inhibitor, which may be either steroidal or non-steroidal. The pharmaceutical composition is in one of the following forms: a vaginal suppository, a rectal suppository, a vaginal gel, a rectal gel, a vaginal cream or a rectal cream. The pharmaceutical composition may optionally comprise pharmaceutically effective amounts of progesterone and calcitriol, and may be administered in combination with Food and Drug Administration (FDA) approved dosage ranges of an oral COX-2 inhibitor, such as rofecoxib, celecoxib, valdecoxib or similar material. Alternatively, the pharmaceutical composition may include an aromatase inhibitor administered vaginally or rectally and is administered in combination with oral calcitriol and oral COX-2 inhibitor. The aromatase inhibitor may be either steroidal or non-steroidal. Steroidal aromatase inhibitors include anastrozole or letrozole, while non-steroidal aromatase inhibitors include exemestane or formestane.

Accordingly, it is a principal object of the invention to provide a method of treating endometriosis in a patient requiring such treatment.

It is another object of the invention to provide a method of treating endometriosis using a vaginal or rectal suppository, cream or gel comprising a pharmaceutically effective amount of an aromatase inhibitor.

It is a further object of the invention to cure severe endometriosis in a patient with severe endometriosis.

It is an object of the invention to provide improved elements and arrangements thereof for the purposes described which is inexpensive, dependable and fully effective in accomplishing its intended purposes.

These and other objects of the present invention will become readily apparent upon further review of the following specification.

### DETAILED DESCRIPTION OF THE PREFERRED EMBODIMENTS

The present invention is directed to treating endometriosis using a vaginal or rectal suppository, cream or gel comprising a pharmaceutically effective amount of a steroidal or non-steroidal aromatase inhibitor.

The endometriosis treatment protocol comprises the step of administering to a female patient in need of such treatment a pharmaceutical composition in a form suitable for vaginal or rectal delivery comprising a pharmaceutically effective amount of the aromatase inhibitor. The pharmaceutical composition is preferably delivered in a vaginal suppository, a rectal suppository, a vaginal gel, a rectal gel, a vaginal cream, or a rectal cream. The pharmaceutical composition may optionally comprise pharmaceutically effective amounts of progesterone and/or calcitriol, and may be administered in combination with FDA-approved dosage ranges of an oral COX-2 inhibitor, such as rofecoxib, celecoxib, valdecoxib or similar material. Alternatively, the pharmaceutical composition comprises an aromatase inhibitor administered vaginally or rectally and is administered in combination with oral calcitriol and the oral COX-2 inhibitor. The aromatase inhibitor may be either steroidal or non-steroidal. Steroidal aromatase inhibitors include anastrozole and letrozole, while non-steroidal aromatase inhibitors include exemestane and formestane.

An effective amount of the aromatase inhibitor in the pharmaceutical composition of the invention may vary, depending on the identity of the aromatase inhibitor and the specific medical condition of the patient. For example, anastrozole may be administered in a dosage range between about 0.1 and 5 mg per vaginal suppository administered one time per day, and preferably at about 1 mg of the aromatase inhibitor anastrozole per vaginal suppository administered one time per day. Alternatively, the aromatase inhibitor letrozole may be administered in a dosage in the range between about 0.1 mg and 10 mg per vaginal suppository administered one time per day, and preferably at about 2.5 mg per vaginal suppository administered one time per day.

The aromatase inhibitor exemestane may be administered in a dosage in the range between about 1 mg and 75 mg per vaginal suppository administered one time per day, and preferably at about 25 mg per vaginal suppository administered one time per day.

### Case Report For Vaginal Suppositories

Patient description: A 47-year-old endometriosis and infertility patient who had chronic pelvic pain and pain during intercourse over a 25 year period. During this time the patient had multiple laparoscopies and traditional medical treatments that provided only temporary relief for a maximum of one to two years. In September 2001, at the age of 45, the patient had a hysterectomy and bilateral salpingo-oophorectomy (total removal of the uterus and both ovaries) to relieve the chronic pelvic pain and pain with intercourse. Because the ovaries were removed, the patient began having many estrogen deficiency symptoms, including: severe hot flashes, mood swings, depression, loss of libido and vaginal dryness. To relieve these symptoms the patient was started on estrogen replacement therapy. However, soon after starting various types of estrogen replacement, symptoms of pelvic pain returned which indicated reactivation of the endometriosis.

After failing all traditional treatment for endometriosis, the patient sought alternative treatment for the control of the disease and hormonal replacement. At the time of her first consultation for alternative treatment, her physical examination showed tender nodularity in the pelvic area and a particularly large tender nodule in the recto-vaginal area, estimated to be approximately 1 cm in diameter. After full evaluation, she agreed to try a new treatment modality to try to shrink the resistant endometriosis nodule and pelvic implants, as well as to reduce pain and inflammation. If she then became stable and pain free, she could re-try hormonal therapy to relieve the estrogen deficiency symptoms.

New Endometriosis Treatment Protocol: In October 2002, the patient was started on treatment with vaginal suppositories comprising 1 mg of anastrozole (a non-steroidal aromatase inhibitor) and progesterone 200 mg. One such suppository was administered nightly per vagina. Additionally, the patient was given Calcitriol 0.5 mcg (Vitamin D active form) 2 capsules and rofecoxib 12.5 mg, both given orally once daily.

Due to the vaginal atrophy secondary to estrogen deficiency, vaginal irritation from the suppositories occurred after six weeks of treatment. Therefore, the suppositories were administered rectally, one suppository at bedtime for six more weeks. This completed a total of three months therapy with the aromatase inhibitor and associated medications.

Examination after treatment showed complete resolution of all of the nodules and tenderness previously found, including the 1 cm nodule in the recto-vaginal area.

Treatment Course: During the first six weeks of treatment there was a steady reduction in pain symptoms, estimated by the patient to be approximately 70% decreased. Pain symptoms, including pain during intercourse, were reduced to zero after three months of treatment.

During the first six weeks of treatment, severe estrogen deficiency symptoms appeared, including night sweats, mood swings, depression and vaginal dryness. After the first six weeks low dose estrogen was, therefore, added in the form of estrogen sublingual drops 1 mg—1 to 3 times daily as needed to control the symptoms. This allowed her to treat with the lowest possible dose that would be less likely to interfere with the suppressive treatment of the endometriosis implants. During the rest of the treatment course, the estrogen sublingual drops controlled most of the symptoms with no return of pelvic pain.

After completion of the three-month treatment protocol, the low dose sublingual drops were changed to a complete hormone replacement regimen: estradiol 1 mg, progesterone 50 mg, and testosterone 2 mg in an alcohol based topical gel daily. There was return of normal mood and libido, and complete relief of all estrogen deficiency symptoms, including vaginal dryness and pain with intercourse. This hormonal replacement has been maintained without the return of any symptoms of endometriosis during the 10-month post-treatment period, a good indication that the endometriosis is remaining in full remission or is permanently cured. Follow-up examinations have shown normalization of the vaginal atrophic changes and lack of any residual nodules of endometriosis.

2. Discussion

The new treatment using vaginal/rectal delivered aromatase inhibitor with progesterone and orally supplemented rofecoxib and Calcitriol achieved exceptional results. The novel approach to deliver the key ingredient, the aromatase inhibitor, directly into the pelvic area where the disease process is localized resulted in an unusually rapid resolution of the endometriosis.

Vaginal and rectal routes of delivery provide effective absorption of drugs and hormones into the vasculature and lymphatics that diffuse these compounds into the area of the disease at higher concentrations than would normally be obtained through the oral route. Additionally, with the oral route, after absorption into the gastrointestinal tract, there is delivery of the compounds directly into the liver where some of the ingredients are metabolized or eliminated, therefore reducing the available amounts of the drugs or hormones for transport through the circulatory system to reach the implants. This phenomenon of liver metabolism or removal is called the “first pass effect”. With the vaginal or rectal routes of absorption, there is a direct delivery into the circulation without the removal of drugs and hormones by the liver through the “first pass effect”. Higher concentrations of the major ingredients reaching the implants might help to explain the unique, rapid clinical response seen in this case. It should be understood that any theory or explanation offered herein to explain the remarkable results achieved in the present invention should not be viewed as limiting the invention in any way, even if the offered explanation or theory turns out to be incorrect.

To date, severe recurrent types of endometriosis are resistant to treatment or cure except for limited periods of time. In the present patient, all previous treatments were ineffective in maintaining remission. Hysterectomy failed to clear up all the implants as evidenced by the quick recurrence of pain and nodularity with hormonal replacement therapy with estrogen after surgery.

With the new treatment protocol the rapidity of clearance of symptoms within 3 months is an unusually rapid positive result compared to other types of known treatments. The non-recurrence of either nodularity an examination or pain of any kind after full hormonal replacement for over 10 months is excellent evidence that the endometriosis is either completely gone or in complete remission, a state that did not occur with any previous traditional treatments.

Conclusion: The new treatment protocol offers women a remarkably effective treatment that curtails symptoms and either places the patient in complete remission or cures the patient of endometriosis.

It is to be understood that the present invention is not limited to the embodiments described above, but encompasses any and all embodiments within the scope of the following claims.

1. A method for treating endometriosis, comprising the step of administering to a female patient in need of such treatment a pharmaceutical composition in a form suitable for vaginal or rectal delivery, the pharmaceutical composition comprising a pharmaceutically effective amount of an aromatase inhibitor.

2. The method of claim 1, wherein the pharmaceutical composition is in a form selected from the group consisting of a vaginal suppository, a rectal suppository, a vaginal gel, a rectal gel, a vaginal cream, and a rectal cream.

3. The method of claim 1, wherein the pharmaceutical composition further comprises pharmaceutically effective amounts of progesterone and calcitriol.

4. The method of claim 1, wherein the pharmaceutical composition further comprises pharmaceutically effective amounts of progesterone.

5. The method of claim 1, further comprising the step of orally administering pharmaceutically effective amounts of calcitriol and a COX-2 inhibitor in combination with the vaginal or rectal delivery of the aromatase inhibitor.

6. The method of claim 1, further comprising the step of orally administering a pharmaceutically effective amount of a COX-2 inhibitor in combination with the vaginal or rectal delivery of the aromatase inhibitor.

7. The method of claim 1, wherein the pharmaceutical composition further comprises pharmaceutically effective amounts of progesterone and calcitriol, and wherein the pharmaceutical composition is in a form selected from the group consisting of a vaginal suppository, a rectal suppository, a vaginal gel, a rectal gel, a vaginal cream and a rectal cream.

8. The method of claim 1, wherein the aromatase inhibitor is steroidal.

9. The method of claim 1, wherein the aromatase inhibitor is non-steroidal.

10. The method of claim 1, wherein the aromatase inhibitor is selected from the group consisting of anastrozole, letrozole, exemestane and formestane.

11. The method of claim 1, wherein the aromatase inhibitor comprises between about 0.1 mg and 0.5 mg anastrozole, and wherein the pharmaceutical composition is in a form selected from the group consisting of a vaginal suppository, a rectal suppository, a vaginal gel, a rectal gel, a vaginal cream and a rectal cream.

12. The method of claim 11, wherein the aromatase inhibitor consists essentially of about 1 mg anastrozole.

13. The method of claim 12, wherein the pharmaceutical composition is administered as a once daily vaginal application.

14. The method of claim 12, wherein the pharmaceutical composition is administered as a once daily rectal application.

15. The method of claim 1, wherein the aromatase inhibitor comprises between about 0.1 mg and 10 mg letrozole and the pharmaceutical composition is in a form selected from the group consisting of a vaginal suppository, a rectal suppository, a vaginal gel, a rectal gel, a vaginal cream and a rectal cream.

16. The method of claim 15, wherein the aromatase inhibitor consists essentially of about 2.5 mg letrozole.

17. The method of claim 15, wherein the pharmaceutical composition is administered as a once daily vaginal application.

18. The method of claim 15, wherein the pharmaceutical composition is administered as a once daily rectal application.

19. The method of claim 1, wherein the aromatase inhibitor comprises between about 1 mg and 75 mg exemestane and the pharmaceutical composition is in a form selected from the group consisting of a vaginal suppository, a rectal suppository, a vaginal gel, a rectal gel, a vaginal cream and a rectal cream.

20. The method of claim 19, wherein the aromatase inhibitor consists essentially of about 25 mg exemestane.

21. The method of claim 19, wherein the pharmaceutical composition is administered as a once daily vaginal application.

22. The method of claim 19, wherein the pharmaceutical composition is administered as a once daily rectal application.

23. A method for treating endometriosis, comprising the step of administering to a female patient in need of such treatment a composition comprising pharmaceutically effective amounts of an aromatase inhibitor, progesterone, and calcitriol, wherein the composition is in the form of a vaginal suppository.

24. A method for treating endometriosis, comprising the step of administering to a female patient in need of such treatment a composition comprising pharmaceutically effective amounts of an aromatase inhibitor and progesterone, wherein the composition is in the form of a vaginal suppository.

25. A pharmaceutical composition for the treatment of endometriosis, comprising:
an effective amount of an aromatase inhibitor;
an effective amount of progesterone; and
an effective amount of calcitriol;
wherein the pharmaceutical composition is in a form selected from the group consisting of a vaginal suppository, a vaginal gel and a vaginal cream.

26. A pharmaceutical composition for the treatment of endometriosis, comprising:
an effective amount of an aromatase inhibitor;
an effective amount of progesterone; and
an effective amount of calcitriol;
wherein the pharmaceutical composition is in a form selected from the group consisting of a rectal suppository, a rectal gel and a rectal cream.

27. A pharmaceutical composition for the treatment of endometriosis, comprising:
an effective amount of an aromatase inhibitor; and
an effective amount of progesterone;
wherein the pharmaceutical composition is in a form selected from the group consisting of a rectal suppository, a rectal gel and a rectal cream.
